(19) United States
(12) Patent Application Publication
(10) Pub. No.: US 2018/0168912 A1
(43) Pub. Date: Jun. 21, 2018

(54) Method for Treatment Using Essential Oil and Stones

(71) Applicant: Kelly Sue-Meisch Larson, Burnsville, MN (US)

(72) Inventor: Kelly Sue-Meisch Larson, Burnsville, MN (US)

(21) Appl. No.: 15/847,488

(22) Filed: Dec. 19, 2017

Related U.S. Application Data
(60) Provisional application No. 62/436,151, filed on Dec. 19, 2016.

(51) Int. Cl. A61H 7/00; A61G 13/00; A61K 36/18

(52) U.S. Cl. CPC A61H 7/001; A61G 13/009; A61K 36/18; A61H 2201/164; A61H 2201/1604; A61H 2201/1614; A61H 2201/10

(57) ABSTRACT

The method for treatment using essential oil and stones uses a patient's known positive energetic-feedback response and the patient's known negative energetic-feedback response to identify therapeutic oils and stones that are attuned to the patient's aura energy field. The known positive energetic-feedback response and the known negative energetic-feedback response are specific responses that are elicited from therapeutic stones that are placed within the patient's aura energy field. In the first step in treating fractures in the patient's aura energy field, a practitioner selects a primary stone, a primary therapeutic oil, a collection of secondary stones, a collection of barrier stones, and a healing stone. The practitioner then arranges the selected stones around the patient's body and advantageously positions the stones around the work area. The practitioner then performs aura fracture identification, cleansing, and sealing processes. These processes cleanse and repair the patient's aura energy field.

The current application claims a priority to the U.S. Provisional Patent application Ser. No. 62/436,151 filed on Dec. 19, 2016.

### FIELD OF THE INVENTION

The present invention relates generally to a method for performing aura energy treatments. More specifically, the present invention relates to a method for repairing aura energy fractures using therapeutic stones and essential oils.

### BACKGROUND OF THE INVENTION

The aura energy fields of humans are constantly interacting with the world. This interaction leads to the formation of relationships and has a marked effect on the quality of a person's health and wellbeing. Because the person is unable to completely control the external aura energy fields with which they come into contact, the person's own aura energy field may become damaged, disrupted, and distorted. Additionally, the person's aura energy field may become damaged, disrupted, and distorted due to internal causes such as unforgiveness or past trauma. This disruption leads to a decreased quality of life for the person and may lead to serious illness.

The present invention, the method for treatment using essential oil and stones, is a treatment routine that cleanses and repairs aura energy fractures. The present invention makes use of therapeutic oils and stones that are attuned to a patient's aura energy field. This enables a skilled practitioner to identify, cleanse, and seal any aura energy fractures that may exist within the patient's aura energy field. By identifying and using therapeutic oils and stones that are attuned to the patient's aura energy field, the practitioner is able to administer an aura energy treatment that is tailored to the patient's unique health requirements.

### BRIEF DESCRIPTION OF THE DRAWINGS

FIG. 1 is a block diagram illustrating the system overview of the present invention.

FIG. 2 is a flowchart describing the overall process followed by the method of the present invention.

FIG. 3 is a flowchart describing a process for selecting the primary stone through the method of the present invention.

FIG. 4 is a flowchart describing a process for selecting the secondary stone types through the method of the present invention.

FIG. 5 is a flowchart describing a process for positioning the plurality of secondary stones through the method of the present invention.

FIG. 6 is a flowchart describing a process for selecting the barrier stone type through the method of the present invention.

FIG. 7 is a flowchart describing a process for positioning the plurality of barrier stones through the method of the present invention.

FIG. 8 is a flowchart describing a process for selecting the healing stone through the method of the present invention.

FIG. 9 is a flowchart describing a process for identifying the aura fracture through the method of the present invention.

FIG. 10 is a flowchart describing a process for selecting the primary oil through the method of the present invention.

FIG. 11 is a flowchart describing a process for reciting the healing therapeutic phrase through the method of the present invention.

FIG. 12 is a flowchart describing a process for cleansing the aura energy fracture through the method of the present invention.

FIG. 13 is a flowchart describing a process for sealing the aura energy fracture through the method of the present invention.

FIG. 14 is a flowchart describing a process for completing the aura-fracture-sealing process through the method of the present invention.

### DETAIL DESCRIPTIONS OF THE INVENTION

All illustrations of the drawings are for the purpose of describing selected versions of the present invention and are not intended to limit the scope of the present invention.

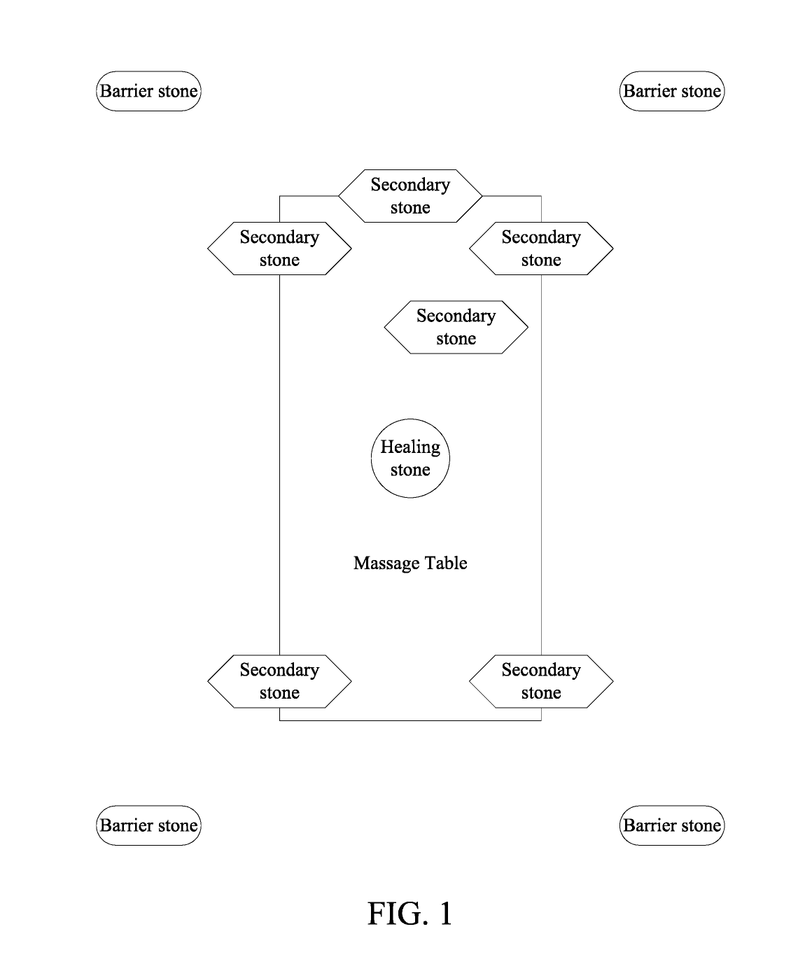

As can be seen in FIG. 1 through FIG. 14, the present invention, the method for treatment using essential oil and stones, is a method used to treat disruptions in a patient's aura energy pathways. The present invention is a method that makes use of a variety of therapeutic stones and a variety of therapeutic oils that are attuned to the patient's aura energy flow. To execute the method of the present invention, a practitioner lays the patient onto a massage table or bed. The practitioner then arranges a variety of therapeutic stones that are attuned to the patient's aura energy field around the patient's body. This creates an environment conducive to aura healing. The practitioner then attaches a therapeutic stone to a pendulum to create an aura energy feedback system. The aura energy feedback system is a device that that detects an energetic-feedback response, which is induced in the therapeutic stone attached to the pendulum, when the therapeutic stone is placed within the patient's aura energy field. The practitioner uses the aura energy feedback system to identify positive and negative energetic-feedback responses that are induced in the therapeutic stone. To continue the treatment, the practitioner suspends the pendulum over various locations of the patient's body to identify fracture points in the patient's aura energy field. Each fracture point is identified when a negative energetic-feedback response is induced in the aura energy feedback system. The practitioner then applies a quantity of therapeutic oil onto each of the fracture points. Once the therapeutic oil has been applied to each fracture point, the practitioner performs a cleansing process to purify the aura fractures. After the aura fractures are cleansed, the practitioner performs a healing process to mend the aura fractures. Once the healing process is completed, the patient's aura energy field are repaired, and the practitioner applies a quantity of essential oil the patient to relieve tension and clear the patient's mind. It is to be understood that the method of the present invention is performed on a patient that is preferably laying prone on a massage table.

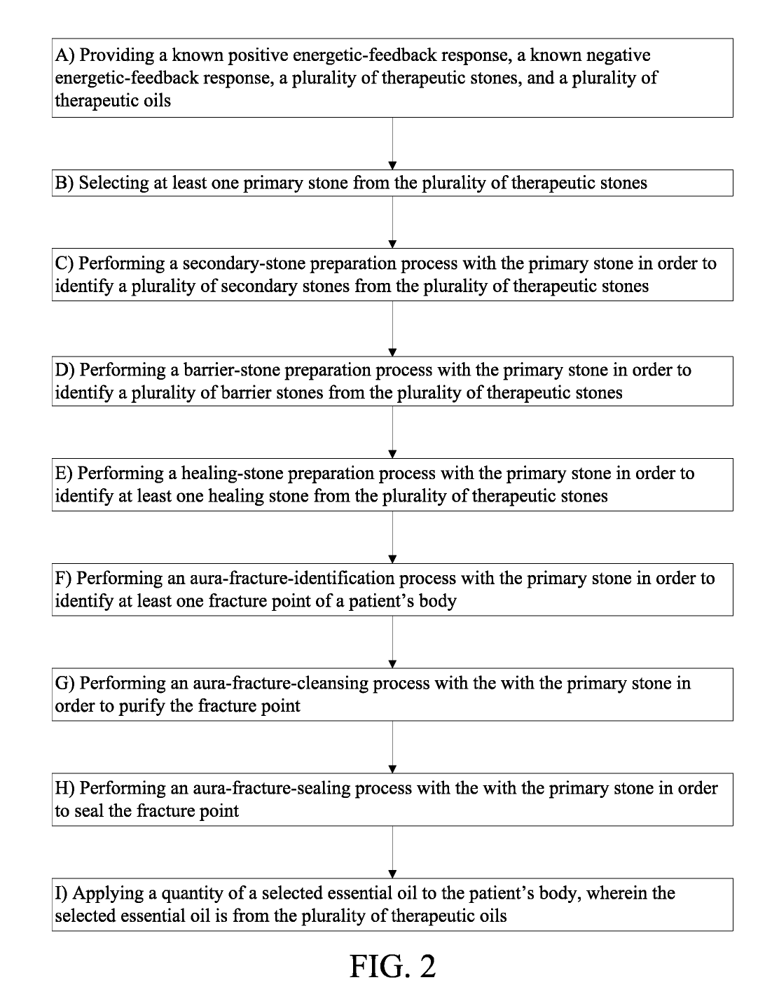

As can be seen in FIG. 2, the system required to perform the above-described process provides a known positive energetic feedback response, a known negative energetic feedback response, a plurality of therapeutic stones, a plurality of therapeutic oils, a pendulum, and a massage table (Step A). The known positive energetic-feedback response and the known negative energetic-feedback response are responses that the patient knows will be induced in the aura energy feedback system then the aura energy feedback system is placed within the patient's aura energy field. Specifically, the positive energetic-feedback response is a specific pattern that is traced by the therapeutic stone attached to the pendulum when the therapeutic stone is placed within an undamaged section of the patient's aura energy field. Similarly, the negative energetic-feedback response is a specific pattern that is traced by the therapeutic stone attached to the pendulum when the therapeutic stone is placed within a damaged section of the patient's aura energy field. The plurality of therapeutic stones includes stones with known therapeutic properties, this group includes, but is not limited to, onyx, hematite, black tourmaline, selenite, and rose quartz. The plurality of therapeutic oils comprises one or more essential oils.

As can be seen in FIG. 2, the overall method of the present invention employs various processes to complete the aura fracture repair process. Specifically, the overall method of the present invention begins by selecting at least one primary stone from the plurality of therapeutic stones (Step B). The primary stone is the therapeutic stone that will be attached to the pendulum of the aura energy feedback system. The user selects the primary stone by attaching therapeutic stones to the pendulum and then suspending each therapeutic stone within the patient's aura energy field. The therapeutic stone in which the positive energetic feedback response is induced is selected as the primary stone. After the primary stone is selected the aura energy feedback system is able to identify substances that react with the patient's aura energy field. Additionally, the aura energy feedback system is able to identify fracture points within the patient's aura energy field.

As can be seen in FIG. 2, once the primary stone is selected the practitioner is ready to begin further preparations for the aura fracture identification, cleansing, and sealing processes. As such, the overall method of the present invention continues by performing a secondary-stone preparation process with the primary stone in order to identify a plurality of secondary stones from the plurality of therapeutic stones (Step C). The secondary-stone preparation process is used to select a group of secondary therapeutic stones that are positioned around the body of the patient during the aura fracture identification, cleansing, and sealing processes. Each of the plurality of secondary stones is selected from the plurality of therapeutic stones and adds a desired therapeutic benefit to the patient during treatment. Specifically, the practitioner uses the aura energy feedback system to identify the plurality of secondary stones that elicit the positive energetic-feedback response from the primary stone. The overall method of the present invention continues by performing a barrier-stone preparation process with the primary stone in order to identify a plurality of barrier stones from the plurality of therapeutic stones (Step D). Similar to the secondary-stone preparation process, the barrier-stone preparation process is used to select a group of barrier stones that are positioned around the working area during the aura fracture identification, cleansing, and sealing processes. Each of the plurality of barrier stones is selected from the plurality of therapeutic stones and adds a desired therapeutic benefit to the patient during treatment. Specifically, the practitioner uses the aura energy feedback system to identify the plurality of barrier stones that elicit the positive energetic-feedback response from the primary stone. The overall method of the present invention continues by performing a healing-stone preparation process with the primary stone in order to identify at least one healing stone from the plurality of therapeutic stones (Step E). Also, similar to the secondary-stone preparation process, the healing-stone preparation process is used to select a healing stone that is positioned above the patient's body during the aura fracture identification, cleansing, and sealing processes. The healing stone is selected from the plurality of therapeutic stones and adds a desired therapeutic benefit to the patient during treatment. Specifically, the practitioner uses the aura energy feedback system to identify the healing stone that elicits the positive energetic-feedback response from the primary stone.

As can be seen in FIG. 2, once the plurality of secondary stones, the plurality of barrier stones, and the healing stone have been selected and arranged, the practitioner begins the process of repairing the patient's aura energy fractures. Specifically, the overall method of the present invention continues by performing an aura-fracture-identification process with the primary stone in order to identify at least one fracture point of a patient's body (Step F). The aura-fracture-identification process is used to identify any fracture points within the patient's aura energy field. Specifically, the practitioner uses the aura energy feedback system to identify at least one location on the patient's body that elicits the negative energetic-feedback response from the primary stone. Additionally, the practitioner applies a quantity of therapeutic oil to the patient's body at the fracture point. This prepares the patient's body and aura energy field for the cleansing, and sealing processes.

As can be seen in FIG. 2, once the quantity of therapeutic oil has been applied to the fracture point, the practitioner begins the process of repairing the patient's aura energy fractures. Specifically, the overall method of the present invention continues by performing an aura-fracture-cleansing process with the primary stone in order to purify the fracture point (Step G). The aura-fracture-cleansing process is used to clear negative energies and emotions that cause fracture points within the patient's aura energy field. Specifically, the practitioner performs a psychospiritual analysis of the patient while suspending the primary stone over the fracture point. The practitioner then uses the aura energy feedback system to identify when the fracture point no longer elicits the negative energetic-feedback response from the primary stone. This prepares the patient's body and aura energy field for the sealing processes.

As can be seen in FIG. 2, once the fracture point has been cleansed, the practitioner begins the process of sealing the patient's aura energy fractures. Specifically, the overall method of the present invention continues by performing an aura-fracture-sealing process with the with the primary stone in order to seal the fracture point (Step H). The aura-fracture-sealing process is used to repair the cleansed fracture point within the patient's aura energy field. Specifically, the practitioner performs a psycholospiritual analysis of the patient while suspending the primary stone over the fracture point. The practitioner then uses the aura energy feedback system to identify when the fracture point elicits the positive energetic-feedback response from the primary stone. This repairs fractures in the patient's aura energy field. Finally, the overall method of the present invention concludes by applying a quantity of a selected essential oil to the patient's body, wherein the selected essential oil is from the plurality of therapeutic oils (Step I). The practitioner massages the patient with the selected oil to promote health and to remove any lightheadedness that may occur after the aura fracture treatment processes. Thus completed, the overall method of the present invention promotes health and wellness.

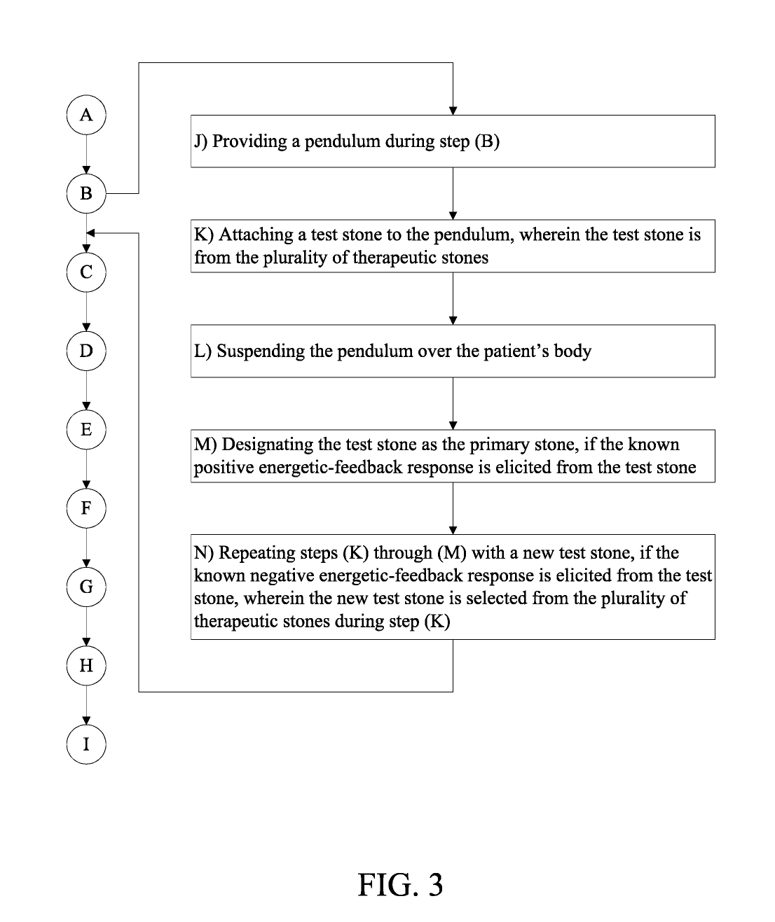

As can be seen in FIG. 3, to fully disclose the steps required to execute the method of the present invention, it is necessary to explain the process of selecting the primary stone from the plurality of therapeutic stones. The process for selecting the primary stone begins by providing a pendulum during Step B (Step J). The process continues by attaching a test stone to the pendulum (Step K). The test stone is a therapeutic stone from the plurality of therapeutic stones that will be tested by being placed within the patient's aura energy field. Additionally, the aura energy feedback system comprises a therapeutic stone and a pendulum. Thus, by attaching the test stone to the pendulum, the user creates an aura energy feedback system. The process continues by suspending the pendulum over the patient's body (step L). Thus suspended, the test stone is placed within the patient's aura energy field. The process continues by designating the test stone as the primary stone, if the known positive energetic-feedback response is elicited from the test stone (step M). The practitioner then watches the test stone for the known positive energetic feedback response. If the known positive energetic-feedback response is observed, then the practitioner is able to determine that the test stone is attuned to the patient's aura energy field. The process continues by repeating Step K through Step M with a new test stone, if the known negative energetic-feedback response is elicited from the test stone (step N). The practitioner is able to repeat the stone selection process for any number of new stones selected from the plurality of therapeutic stones during Step K. Once the practitioner identifies a test stone that is attuned to the patient's aura energy field, the practitioner designates the test stone as the primary stone, and uses the primary stone when performing the remaining processes of the method of the present invention.

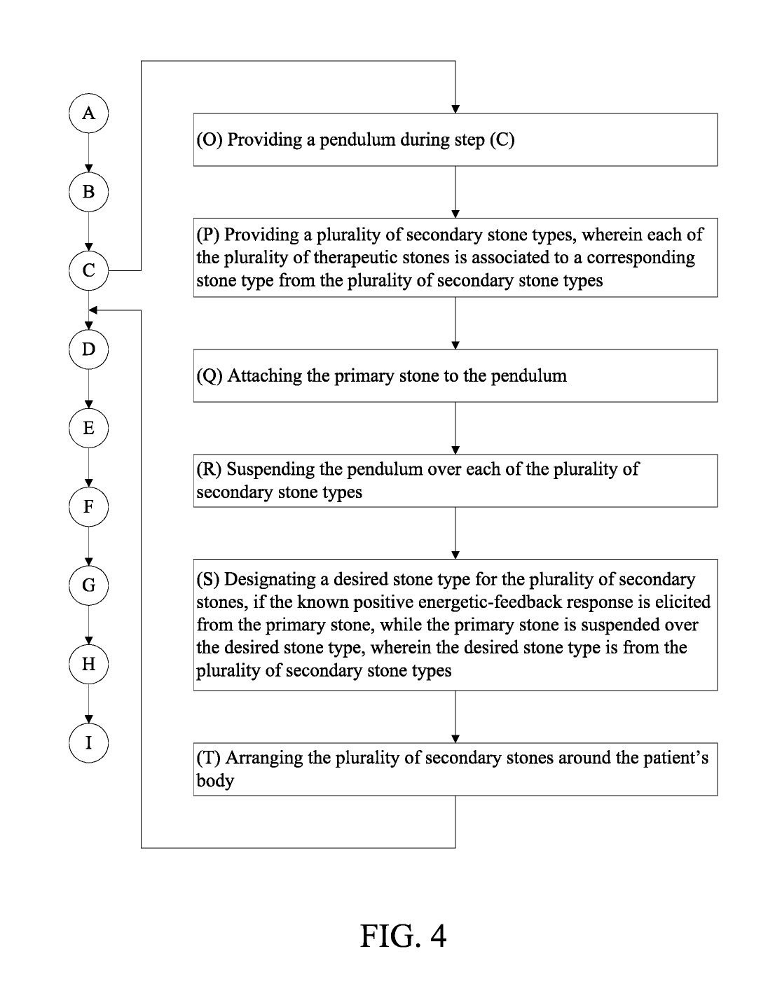

As can be seen in FIG. 4, to fully disclose the steps required to execute the method of the present invention, it is necessary to explain the secondary-stone preparation process in greater detail. The process for selecting the plurality of secondary stones begins by providing a pendulum during Step C (Step O). The process continues by providing a plurality of secondary stone types (Step P). Each of the plurality of secondary stone types relates to a specific category of stone e.g. diamonds, emeralds, and quartz. Additionally, each of the plurality of therapeutic stones is associated to a corresponding stone type from the plurality of secondary stone types. Accordingly, the practitioner only has to identify a specific type of stone that is attuned to the patient's aura energy field when selecting the plurality of secondary stones. The process continues by attaching a primary stone to the pendulum (Step Q). The process continues by suspending the pendulum over each of the plurality of secondary stone types (step R). Thus suspended, the primary stone is sequentially placed within the aura energy field of each of the plurality of secondary stone types. The process continues by designating a desired stone type for the plurality of secondary stones, if the known positive energetic-feedback response is elicited from the primary stone, while the primary stone is suspended over the desired stone type (step S). The practitioner then watches the primary stone for the known positive energetic feedback response. If the known positive energetic-feedback response is observed, then the practitioner is able to determine that the desired stone type is attuned to the patient's aura energy field. Furthermore, the desired stone type is from the plurality of secondary stone types. Finally, the process concludes by arranging the plurality of secondary stones around the patient's body. The practitioner positions each of the plurality of secondary stones at a location around the patient's body that facilitates performing the aura fracture identification, cleansing, and sealing processes.

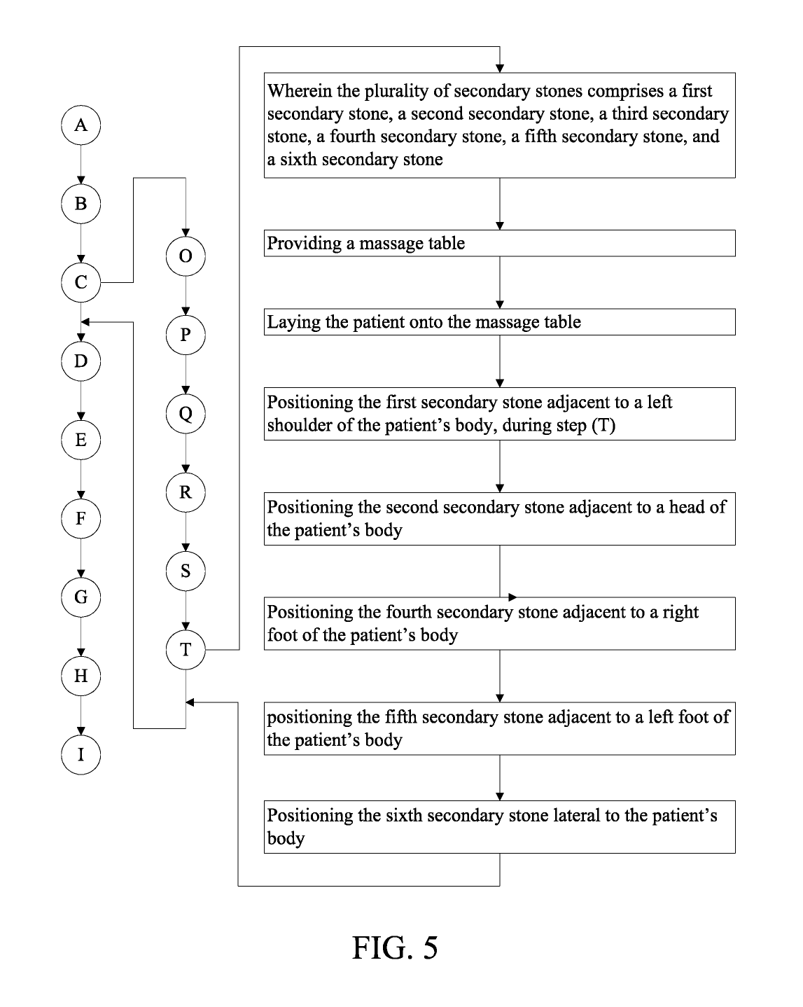

As can be seen in FIG. 5, in the preferred method of the present invention the plurality of secondary stones comprises a first secondary stone, a second secondary stone, a third secondary stone, a fourth secondary stone, a fifth secondary stone, and a sixth secondary stone. Additionally, each of the plurality of secondary stones is positioned in adjacent to a specific body part. To begin preparing the patient for the aura fracture identification, cleansing, and sealing processes, the practitioner first lays the patient onto the massage table. The practitioner then positions the first secondary stone adjacent to a left shoulder of the patient's body, during step. Next, the practitioner positions the second secondary stone adjacent to a head of the patient's body. Similarly, the practitioner then positions the third secondary stone adjacent to a right shoulder of the patient's body. Likewise, the practitioner then positions the fourth secondary stone adjacent to a right foot of the patient's body. Further, the practitioner then positions the fifth secondary stone adjacent to a left foot of the patient's body. Finally, the practitioner then positions the sixth secondary stone lateral to the patient's body (Step T). Thus positioned, the plurality of secondary stones facilitates performing the aura fracture identification, cleansing, and sealing processes.

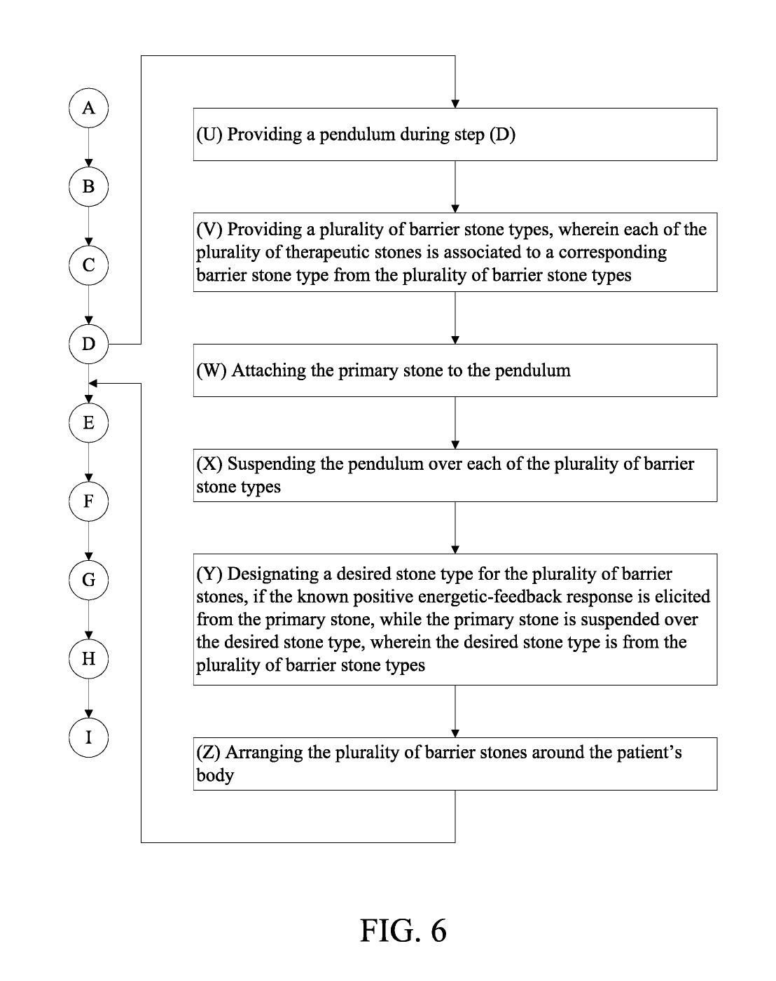

As can be seen in FIG. 6, to fully disclose the steps required to execute the method of the present invention, it is necessary to explain the barrier-stone preparation process in greater detail. The process for selecting the plurality of barrier stones begins by providing a pendulum during Step D (Step U). The process continues by providing a plurality of barrier stone types (Step V). Each of the plurality of barrier stone types relates to a specific category of stone e.g. diamonds, emeralds, and quartz. Additionally, each of the plurality of therapeutic stones is associated to a corresponding stone type from the plurality of barrier stone types. Accordingly, the practitioner only has to identify a specific type of stone that is attuned to the patient's aura energy field when selecting the plurality of barrier stones. The process continues by attaching a primary stone to the pendulum (Step W). The process continues by suspending the pendulum over each of the plurality of barrier stone types (step X). Thus suspended, the primary stone is sequentially placed within the aura energy field of each of the plurality of barrier stone types. The process continues by designating a desired stone type for the plurality of barrier stones, if the known positive energetic-feedback response is elicited from the primary stone, while the primary stone is suspended over the desired stone type (step Y). The practitioner then watches the primary stone for the known positive energetic feedback response. If the known positive energetic-feedback response is observed, then the practitioner is able to determine that the desired stone type is attuned to the patient's aura energy field. Furthermore, the desired stone type is from the plurality of barrier stone types. Finally, the process concludes by arranging the plurality of barrier stones around the patient's body (Step Z). The practitioner positions each of the plurality of barrier stones at a location around the patient's body that facilitates performing the aura fracture identification, cleansing, and sealing processes.

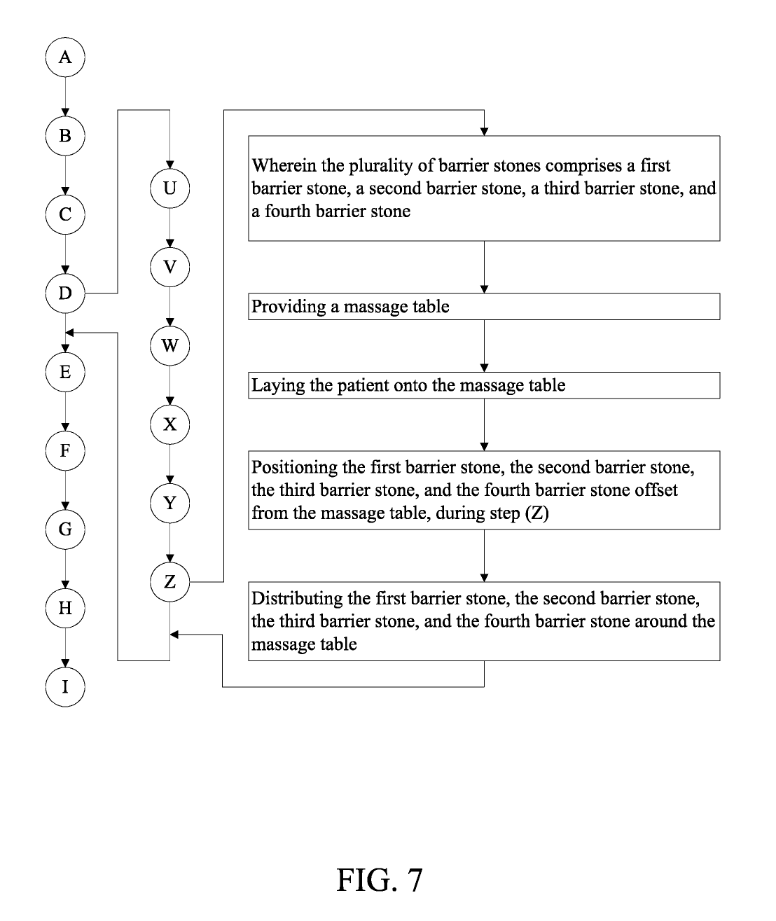

As can be seen in FIG. 7, in the preferred method of the present invention the plurality of barrier stones comprises a first barrier stone, a second barrier stone, a third barrier stone, and a fourth barrier stone. Additionally, each of the plurality of barrier stones is perimetrically around the area being used to perform aura fracture repair. To begin preparing the patient for the aura fracture identification, cleansing, and sealing processes, the patient must be laying on the massage table. The practitioner then positions the first barrier stone, the second barrier stone, the third barrier stone, and the fourth barrier stone offset from the massage table. Additionally, the practitioner distributes the first barrier stone, the second barrier stone, the third barrier stone, and the fourth barrier stone around the massage table, during Step Z. Thus positioned, the plurality of secondary stones facilitates performing the aura fracture identification, cleansing, and sealing processes.

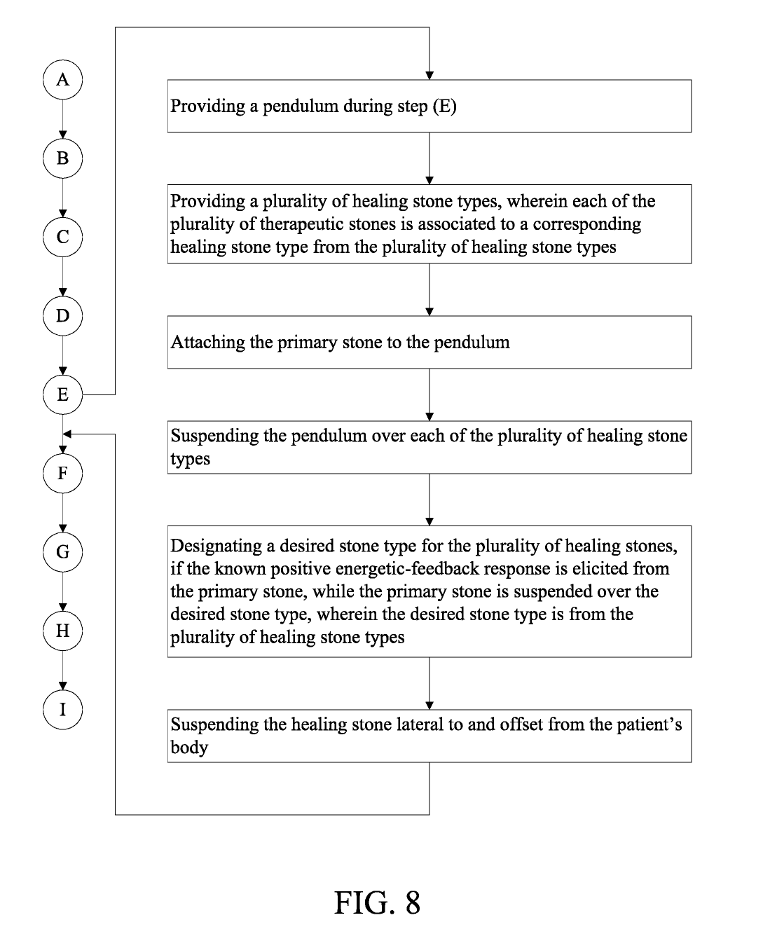

As can be seen in FIG. 8, to fully disclose the steps required to execute the method of the present invention, it is necessary to explain the healing-stone preparation process in greater detail. The process for selecting the healing stone begins by providing a pendulum during Step E. The process continues by providing a plurality of healing stone types. Each of the plurality of healing stone types relates to a specific category of stone e.g. diamonds, emeralds, and quartz. Additionally, each of the plurality of therapeutic stones is associated to a corresponding stone type from the plurality of healing stone types. The process continues by attaching the primary stone to the pendulum. The process continues by suspending the pendulum over each of the plurality of healing stone types. Thus suspended, the primary stone is sequentially placed within the aura energy field of each of the plurality of healing stone types. The process continues by designating a desired stone type for the healing stone, if the known positive energetic-feedback response is elicited from the primary stone, while the primary stone is suspended over the desired stone type. The practitioner then watches the primary stone for the known positive energetic-feedback response. If the known positive energetic-feedback response is observed, then the practitioner is able to determine that the desired stone type is attuned to the patient's aura energy field. Furthermore, the desired stone type is from the plurality of healing stone types. Finally, the process concludes by suspending the healing stone lateral to and offset from the patient's body. The practitioner positions the healing stone at a location that facilitates performing the aura fracture identification, cleansing, and sealing processes.

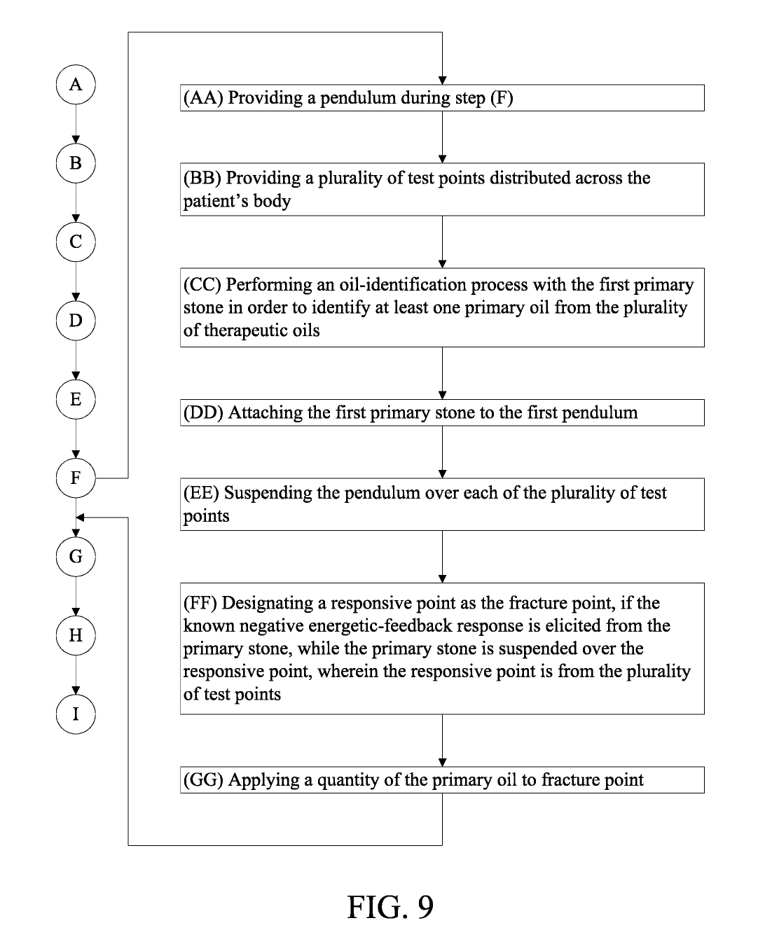

As can be seen in FIG. 9, the aura-fracture-identification process is used to prepare the patient's body and aura energy field for treatment. Accordingly, the aura-fracture-identification process begins by providing a pendulum during Step F (Step AA). The aura-fracture-identification process continues by providing a plurality of test points distributed across the patient's body (Step BB). The plurality of test points is a collection of locations on the human body that are known to be loci for aura energy pathways. The aura-fracture-identification process continues by performing an oil-identification process with the first primary stone in order to identify at least one primary oil from the plurality of therapeutic oils (Step CC). The oil identification process is used to identify a therapeutic oil that is attuned to the patient's aura energy field. This process enables the practitioner to select the appropriate primary oil to treat the disruptions in the patient's aura energy field. The aura-fracture-identification process continues by attaching the primary stone to the pendulum (Step DD). The process continues by suspending the pendulum over each of the plurality of test points (Step EE). Thus suspended, the primary stone is sequentially placed within the field of influence for each of the plurality of test points. The aura-fracture-identification process continues by designating a responsive point as the fracture point, if the known negative energetic-feedback response is elicited from the primary stone, while the primary stone is suspended over the responsive point (Step FF). The practitioner then watches the primary stone for the known negative energetic-feedback response. If the known negative energetic-feedback response is observed, then the practitioner is able to determine that the patient's aura energy field is fractured at the responsive point; this identifying a fracture point in the patient's aura energy field. Furthermore, the responsive point is from the plurality of test points. Finally, the process concludes by applying a quantity of the primary oil to fracture point (Step GG). Accordingly, the fracture point is identified and the quantity of the primary oil begins preparing the patient's body and aura energy field for treatment.

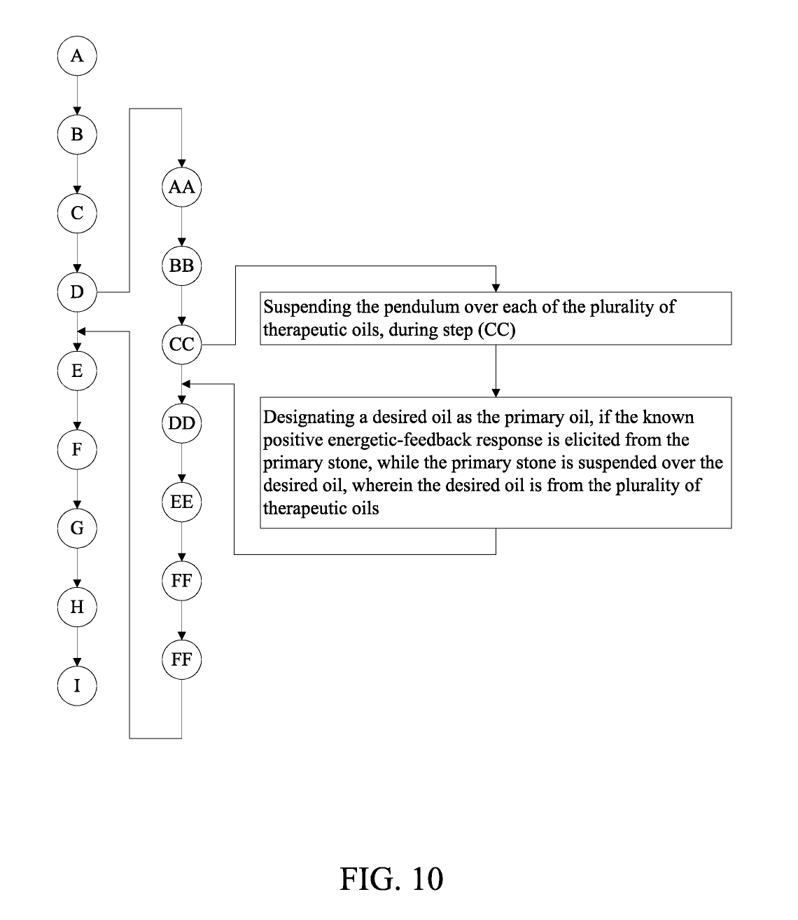

As can be seen in FIG. 10, the oil-identification process is performed in a manner similar to the secondary-stone-identification process. As such, the oil-identification process begins by suspending the pendulum over each of the plurality of therapeutic oils, during step (CC). Thus suspended, the primary stone is sequentially placed within the aura energy field of each of the plurality of therapeutic oils. The oil-identification process continues by designating a desired oil as the primary oil, if the known positive energetic-feedback response is elicited from the primary stone, while the primary stone is suspended over the desired oil. The practitioner then watches the primary stone for the known positive energetic-feedback response. If the known positive energetic-feedback response is observed, then the practitioner is able to determine that the desired oil is attuned to the patient's aura energy field. Furthermore, the desired oil is from the plurality of therapeutic oils. Thus, the practitioner is able to select the most appropriate therapeutic oil for the patient's unique aura energy field.

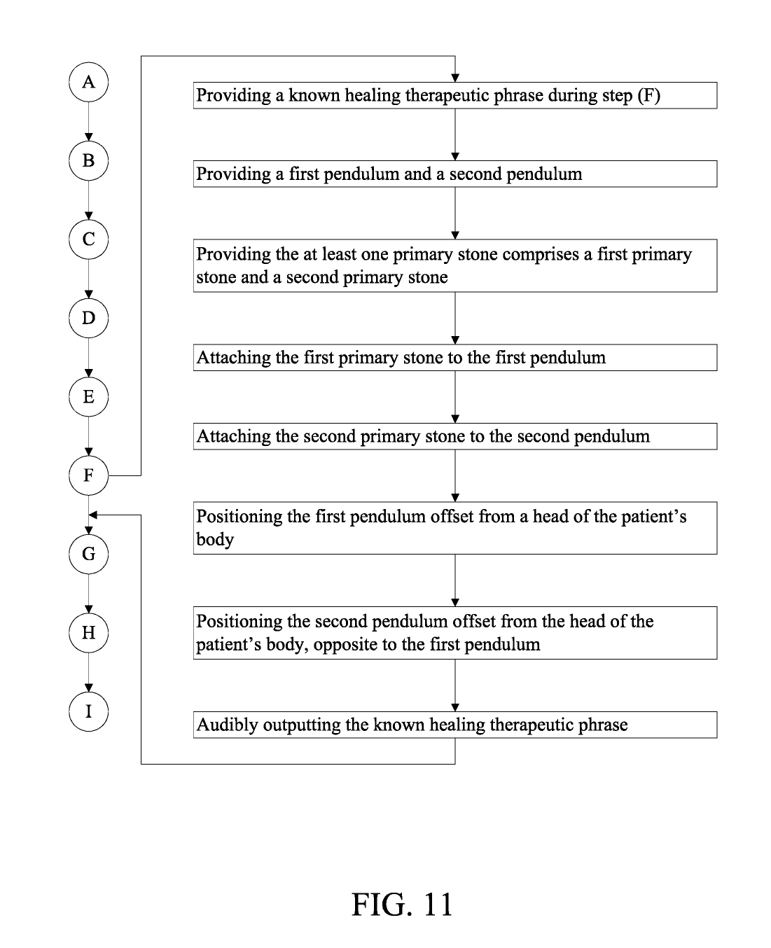

As can be seen in FIG. 11, in addition to identifying the fracture point, the practitioner recites therapeutic phrases during the aura-fracture-identification process. As such, the practitioner must be provided with a known healing therapeutic phrase. The known healing therapeutic phrase is preferably a request for assistance from entities capable of repairing aura energy fractures. Additionally, the practitioner is provided with a first pendulum, a second pendulum, a first primary stone, and a second primary stone. Prior to uttering the therapeutic phrase, the practitioner must attach the first primary stone and the second primary stone to the first pendulum and the second pendulum, respectively. Thus, providing the practitioner with two aura energy feedback systems. The two aura energy feedback systems become focal points for therapeutic energies. To direct the therapeutic energies that are provided by the entities to whom the known healing therapeutic phrase was directed, the practitioner must position the first pendulum offset from a head of the patient's body. Additionally, the practitioner must position the second pendulum offset from the head of the patient's body, opposite to the first pendulum. Thus positioned, the therapeutic energy that is being focused on the first primary stone and the second primary stone is able to beneficially influence the patient's aura energy field.

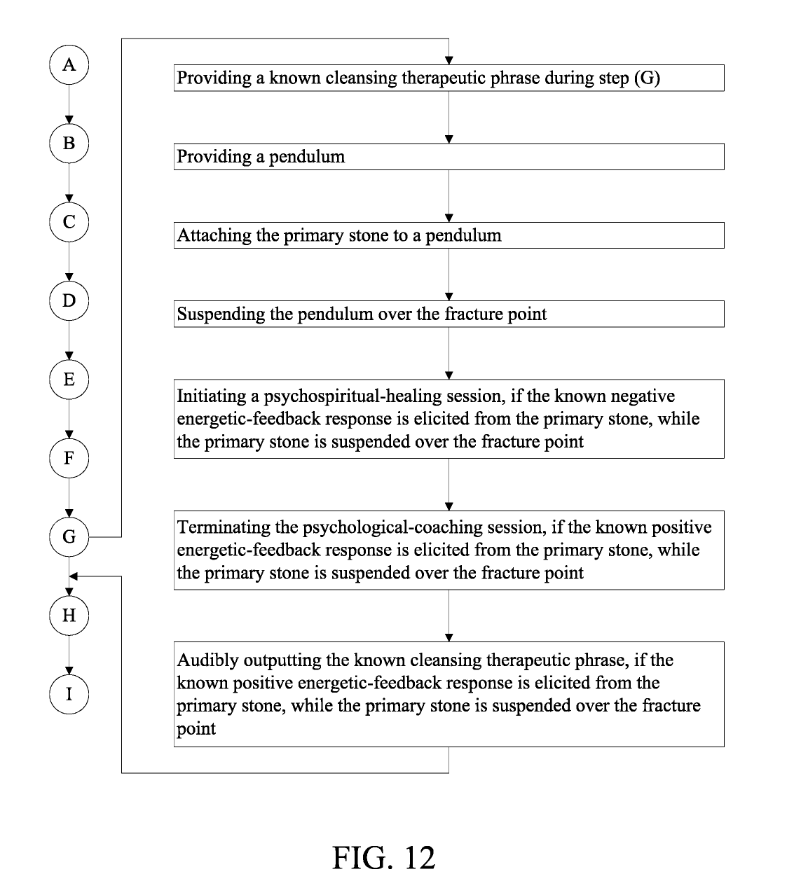

As can be seen in FIG. 12, the aura-fracture-cleansing process is used to identify and remove the causes of fractures in the patient's aura energy field. Accordingly, the aura-fracture-cleansing process begins by providing a known cleansing therapeutic phrase during Step G. The known cleansing therapeutic phrase is preferably a request for assistance from entities capable of cleansing aura energy fractures. The aura-fracture-cleansing process continues by providing a pendulum. The aura-fracture-cleansing process continues by attaching the primary stone to the pendulum. The process continues by suspending the pendulum over the fracture point. Thus suspended, the primary stone and the aura feedback system are able to act as a gauge for changes in the aura energy field around the fracture point. The aura-fracture-cleansing process continues by initiating a psychospiritual-healing session, if the known negative energetic-feedback response is elicited from the primary stone, while the primary stone is suspended over the fracture point. The psychospiritual-healing session is preferably a question and answer session in which the practitioner probes the patient to determine the source of the fracture point. Additionally. The psychospiritual-healing session enables the practitioner to guide the patient through the process of removing the source of the fracture point. The practitioner then watches the primary stone for the known negative energetic-feedback response. If the known negative energetic-feedback response is observed, then the practitioner is able to determine that the patient's aura energy field remains fractured at the fracture point. The practitioner then continues the psychospiritual-healing session. The aura-fracture-cleansing process continues by terminating the psychological-coaching session, if the known positive energetic-feedback response is elicited from the primary stone, while the primary stone is suspended over the fracture point. Accordingly. The practitioner is able to determine that the fracture point has been repaired when the primary stone ceases to exhibit the known negative energetic-feedback response, and begins exhibiting the known positive energetic-feedback response. The aura-fracture-cleansing process continues by audibly outputting the known cleansing therapeutic phrase, if the known positive energetic-feedback response is elicited from the primary stone, while the primary stone is suspended over the fracture point. As a result, the entities, to whom the known cleansing therapeutic phrase was directed, are given the opportunity to assist in the cleansing of the fracture point.

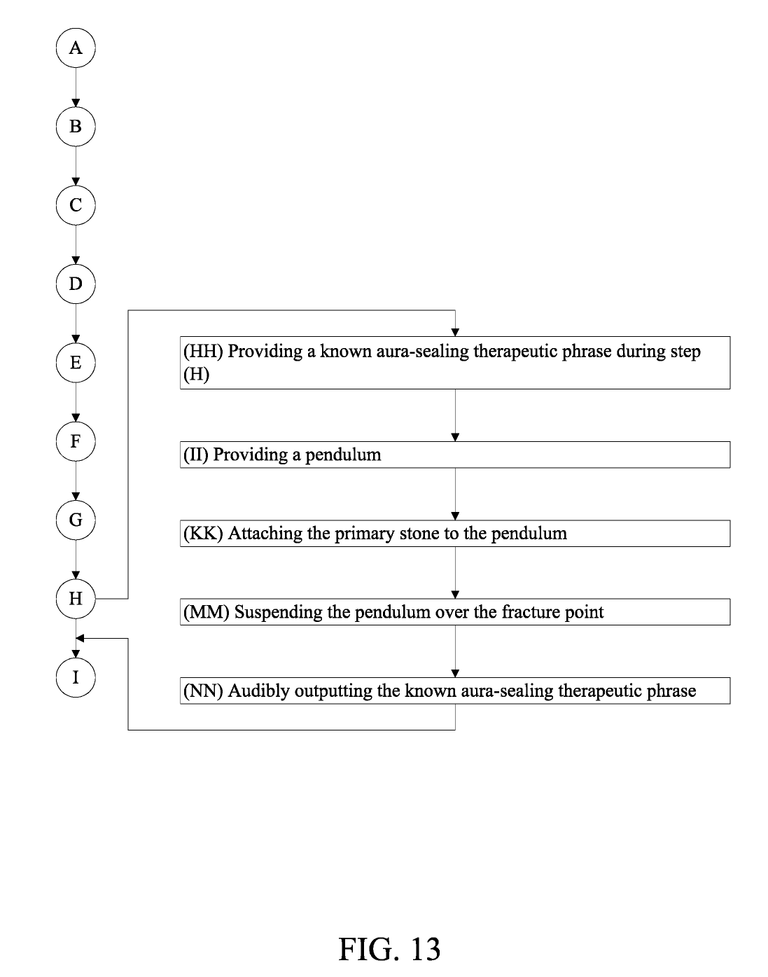

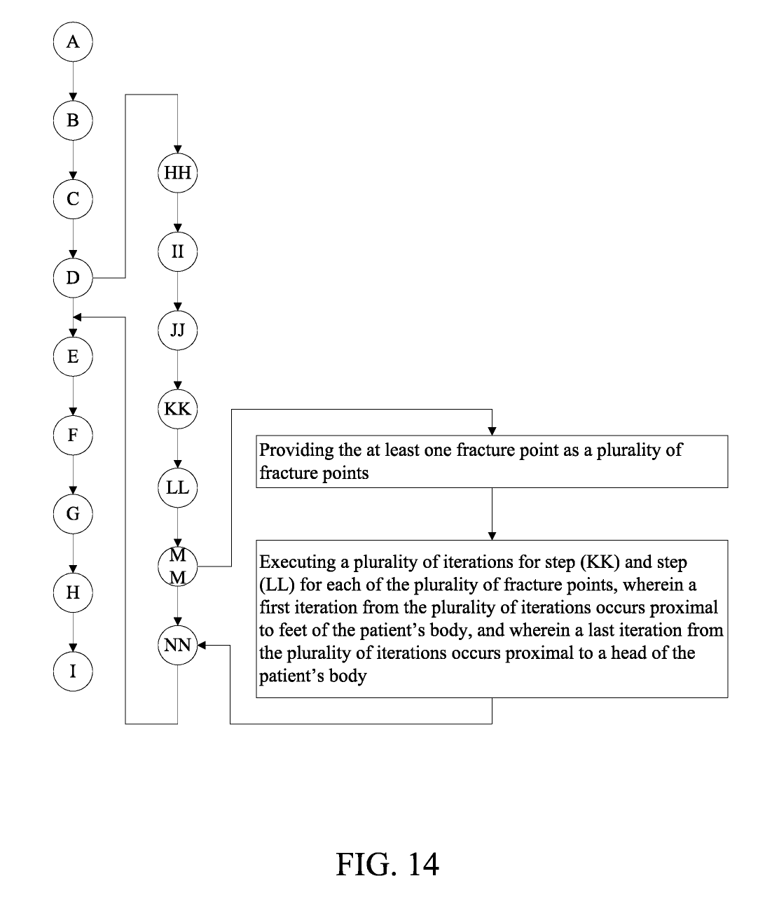

As can be seen in FIG. 13 and FIG. 14, the aura-fracture-sealing process is used to seal the recently cleansed fracture points in the patient's aura energy field. Accordingly, the aura-fracture-sealing process begins by providing a known aura-sealing therapeutic phrase during Step H (Step HH). The known aura-sealing therapeutic phrase is preferably a request for assistance from entities capable of sealing aura energy fractures. The aura-fracture-sealing process continues by providing a pendulum (Step II). The aura-fracture-sealing process continues by attaching the primary stone to the pendulum (Step JJ). The process continues by suspending the pendulum over the fracture point (Step KK). Thus suspended, the primary stone and the aura feedback system are able to act as a gauge for changes in the aura energy field around the fracture point. The aura-fracture-sealing process continues by audibly outputting the known sealing therapeutic phrase (Step LL). As a result, the entities, to whom the known aura-sealing therapeutic phrase was directed, are given the opportunity to assist in the sealing of the fracture point. The aura-fracture-sealing process preferably starts at the fracture point located proximal to the patient's feet. The practitioner then repeats the aura-fracture-sealing process for any remaining fracture points. Specifically, a plurality of iterations for Step KK and Step LL for each of the plurality of fracture points is performed, wherein a first iteration from the plurality of iterations occurs proximal to feet of the patient's body, and wherein a last iteration from the plurality of iterations occurs proximal to a head of the patient's body. As a result, the practitioner is able to seal all of the fracture points that were identified and cleansed.

Although the invention has been explained in relation to its preferred embodiment, it is to be understood that many other possible modifications and variations can be made without departing from the spirit and scope of the invention as hereinafter claimed.

### SUPPLEMENTAL DESCRIPTIONS OF THE INVENTION

The present invention will heal aura fractures and remove negative energy attachments. The process is a form of energy healing. The present invention may assist with:
Release of negative energies and attachments;
Release of bottled emotions;
Mending of aura fractures;
Increased energy flow;
Clearing of mind to increase creativity;
Emotional balance; and
A variety of other energetic responses.

The steps of how the present invention works are as follows:
1. It starts with the selection of 2 pendulums based on energetic feedback from the pendulum.
2. The first chosen pendulum is then used to select essential oils based on a positive energetic response to the oils. One or more essential oils may be chosen. The essential oils chosen are a direct feedback of the client's energetic aura field needs.
3. Next the second pendulum is used to select 6 or more stones and or crystals based on the energetic response of the pendulum. The stones/crystals chosen are a direct feedback of the client's energetic aura field needs.
4. Four stones are chosen for an energetic field barrier and are placed at the four corners of the work space to be used. These stones should be made of Onyx, Hematite or Black Tourmaline. This is to prevent the interference of energetic fields outside of the work space and contain any released negative energy from moving outside of the work space. A healing stone such as selenite or rose quartz should be hung above the middle of the massage table. This will create a pyramid shape between the 5 combined stones.
5. The stones and crystals are positioned around the client at the four corners of the massage table, under the head and under the heart area. If more than 6 stones are chosen, they may be placed based on energetic feedback of the pendulum.
6. The first pendulum is then used to scan over the client's body while holding the essential oil. Upon a positive response from the pendulum a drop of the essential oil should be placed on that section of the client's body. This should be repeated for each essential oil chosen.
7. Next taking a pendulum in each hand and standing at the clients head a short request to the universe will be made stating “I ask the Universal Healing Powers and Energies to work with [name] totems and guides to heal the fractures in their aura and remove all negative energies, entities and attachments and send them to a location appropriate their deeds.”
8. Now using the pendulums scan over the client's body, staying over the head until the pendulum no longer rotates or directs you to another area. Following this process over the entire body and aura field until the entire energetic field of the aura produces a positive response from the pendulum. As the pendulum finds energetic fractures in the aura ask questions of the client that may pertain to the reason that energy may be low in that area such as injury or other emotional reasons. Coaching the client to release any negative energy no longer needed and to welcome in only that energy that is for their highest good. Repeat as needed over severe negative areas the request to the universe stating “I ask the Universal Healing Powers and Energies to work with [name] totems and guides to heal the fractures in their aura and remove all negative energies, entities and attachments and send them to a location appropriate their deeds.”
9. Once this the aura energetic fractures and negative energy release is completed move to the feet and ask the universe to seal the client's aura by stating “I ask the Universal Healing Powers and Energies and [name] totems and guides to seal [name] aura protecting it from future negative energies, entities and attachments.” Working back up the body from the feet to the head stay over each area of the body until the pendulum stops rotating in each area or indicates to move to the next section based on the energetic response of the aura field and pendulums.
10. Once the aura sealing is completed, have the customer sit up slowly and apply a grounding essential oils such as an herbal or tree based essential oil or oil blend to the back of their neck to help counteract any lightheaded feelings.
11. The process is now complete.

What is claimed is:

1. A method for treatment using essential oil and stones comprises the steps of:
(A) providing a known positive energetic-feedback response, a known negative energetic-feedback response, a plurality of therapeutic stones, and a plurality of therapeutic oils;
(B) selecting at least one primary stone from the plurality of therapeutic stones;
(C) performing a secondary-stone preparation process with the primary stone in order to identify a plurality of secondary stones from the plurality of therapeutic stones;
(D) performing a barrier-stone preparation process with the primary stone in order to identify a plurality of barrier stones from the plurality of therapeutic stones;
(E) performing a healing-stone preparation process with the primary stone in order to identify at least one healing stone from the plurality of therapeutic stones;
(F) performing an aura-fracture-identification process with the primary stone in order to identify at least one fracture point of a patient's body;
(G) performing an aura-fracture-cleansing process with the with the primary stone in order to purify the fracture point;
(H) performing an aura-fracture-sealing process with the with the primary stone in order to seal the fracture point;
(I) applying a quantity of a selected essential oil to the patient's body, wherein the selected essential oil is from the plurality of therapeutic oils;

2. The method for treatment using essential oil and stones, as claimed in claim 1, comprises:
(J) providing a pendulum during step (B);
(K) attaching a test stone to the pendulum, wherein the test stone is from the plurality of therapeutic stones;
(L) suspending the pendulum over the patient's body;
(M) designating the test stone as the primary stone, if the known positive energetic-feedback response is elicited from the test stone;
(N) repeating steps (K) through (M) with a new test stone, if the known negative energetic-feedback response is elicited from the test stone, wherein the new test stone is selected from the plurality of therapeutic stones during step (K);

3. The method for treatment using essential oil and stones, as claimed in claim 1, comprises:
(O) providing a pendulum during step (C);
(P) providing a plurality of secondary stone types, wherein each of the plurality of therapeutic stones is associated to a corresponding stone type from the plurality of secondary stone types;
(Q) attaching the primary stone to the pendulum;
(R) suspending the pendulum over each of the plurality of secondary stone types;
(S) designating a desired stone type for the plurality of secondary stones, if the known positive energetic-feedback response is elicited from the primary stone, while the primary stone is suspended over the desired stone type, wherein the desired stone type is from the plurality of secondary stone types;
(T) arranging the plurality of secondary stones around the patient's body;

4. The method for treatment using essential oil and stones, as claimed in claim 3, comprises:
wherein the plurality of secondary stones comprises a first secondary stone, a second secondary stone, a third secondary stone, a fourth secondary stone, a fifth secondary stone, and a sixth secondary stone;
providing a massage table;
laying the patient onto the massage table;
positioning the first secondary stone adjacent to a left shoulder of the patient's body, during step (T);
positioning the second secondary stone adjacent to a head of the patient's body;
positioning the third secondary stone adjacent to a right shoulder of the patient's body;
positioning the fourth secondary stone adjacent to a right foot of the patient's body;
positioning the fifth secondary stone adjacent to a left foot of the patient's body;
positioning the sixth secondary stone lateral to the patient's body;

5. The method for treatment using essential oil and stones, as claimed in claim 1, comprises:
(U) providing a pendulum during step (D);
(V) providing a plurality of barrier stone types, wherein each of the plurality of therapeutic stones is associated to a corresponding barrier stone type from the plurality of barrier stone types;
(W) attaching the primary stone to the pendulum;
(X) suspending the pendulum over each of the plurality of barrier stone types;
(Y) designating a desired stone type for the plurality of barrier stones, if the known positive energetic-feedback response is elicited from the primary stone, while the primary stone is suspended over the desired stone type, wherein the desired stone type is from the plurality of barrier stone types;
(Z) arranging the plurality of barrier stones around the patient's body;

6. The method for treatment using essential oil and stones, as claimed in claim 5, comprises:
wherein the plurality of barrier stones comprises a first barrier stone, a second barrier stone, a third barrier stone, and a fourth barrier stone;
providing a massage table;
laying the patient onto the massage table;
positioning the first barrier stone, the second barrier stone, the third barrier stone, and the fourth barrier stone offset from the massage table, during step (Z);
distributing the first barrier stone, the second barrier stone, the third barrier stone, and the fourth barrier stone around the massage table;

7. The method for treatment using essential oil and stones, as claimed in claim 1, comprises:
providing a pendulum during step (E);
providing a plurality of healing stone types, wherein each of the plurality of therapeutic stones is associated to a corresponding healing stone type from the plurality of healing stone types;
attaching the primary stone to the pendulum;
suspending the pendulum over each of the plurality of healing stone types;
designating a desired stone type for the plurality of healing stones, if the known positive energetic-feedback response is elicited from the primary stone, while the primary stone is suspended over the desired stone type, wherein the desired stone type is from the plurality of healing stone types;
suspending the healing stone lateral to and offset from the patient's body;

8. The method for treatment using essential oil and stones, as claimed in claim 1, comprises:
(AA) providing a pendulum during step (F);
(BB) providing a plurality of test points distributed across the patient's body;
(CC) performing an oil-identification process with the first primary stone in order to identify at least one primary oil from the plurality of therapeutic oils;
(DD) attaching the first primary stone to the pendulum;
(EE) suspending the pendulum over each of the plurality of test points;
(FF) designating a responsive point as the fracture point, if the known negative energetic-feedback response is elicited from the primary stone, while the primary stone is suspended over the responsive point, wherein the responsive point is from the plurality of test points;
(GG) applying a quantity of the primary oil to fracture point;

9. The method for treatment using essential oil and stones, as claimed in claim 8, comprises:
suspending the pendulum over each of the plurality of therapeutic oils, during step (CC);
designating a desired oil as the primary oil, if the known positive energetic-feedback response is elicited from the primary stone, while the primary stone is suspended over the desired oil, wherein the desired oil is from the plurality of therapeutic oils;

10. The method for treatment using essential oil and stones, as claimed in claim 1, comprises:
providing a known healing therapeutic phrase during step (F);
providing a first pendulum and a second pendulum;
providing the at least one primary stone comprises a first primary stone and a second primary stone;
attaching the first primary stone to the first pendulum;
attaching the second primary stone to the second pendulum;
positioning the first pendulum offset from a head of the patient's body;
positioning the second pendulum offset from the head of the patient's body, opposite to the first pendulum;
audibly outputting the known healing therapeutic phrase;

11. The method for treatment using essential oil and stones, as claimed in claim 1, comprises:
providing a known cleansing therapeutic phrase during step (G);
providing a pendulum;
attaching the primary stone to a pendulum;
suspending the pendulum over the fracture point;
initiating a psychospiritual-healing session, if the known negative energetic-feedback response is elicited from the primary stone, while the primary stone is suspended over the fracture point;
terminating the psychological-coaching session, if the known positive energetic-feedback response is elicited from the primary stone, while the primary stone is suspended over the fracture point;
audibly outputting the known cleansing therapeutic phrase, if the known positive energetic-feedback response is elicited from the primary stone, while the primary stone is suspended over the fracture point;

12. The method for treatment using essential oil and stones, as claimed in claim 1, comprises:
(HH) providing a known aura-sealing therapeutic phrase during step (H);
(II) providing a pendulum;
(JJ) attaching the primary stone to the pendulum;
(KK) suspending the pendulum over the fracture point;
(LL) audibly outputting the known aura-sealing therapeutic phrase;

13. The method for treatment using essential oil and stones, as claimed in claim 12, comprises:
providing the at least one fracture point as a plurality of fracture points;
executing a plurality of iterations for step (KK) and step (LL) for each of the plurality of fracture points, wherein a first iteration from the plurality of iterations occurs proximal to feet of the patient's body, and wherein a last iteration from the plurality of iterations occurs proximal to a head of the patient's body;
